(19) United States
(12) Patent Application Publication
(10) Pub. No.: US 2011/0275917 A1
(43) Pub. Date: Nov. 10, 2011

(54) ELECTRORESPONSIVE DEVICE FOR EXTENDING THE LIFE OF BIOSENSORS, AND A BIOSENSOR EMPLOYING THE SAME

(75) Inventors: Sun Ill Kim, Seoul (KR); Seon Jeong Kim, Seoul (KR); Jang Hyun Youn, Cypress, CA (US)

(73) Assignee: Industry-University Cooperation Foundation Hanyang University, Seoul (KR)

(21) Appl. No.: 13/143,960

(22) PCT Filed: Jan. 15, 2010
(86) PCT No.: PCT/KR2010/000279

§ 371 Date: Jul. 11, 2011

(30) Foreign Application Priority Data
Jan. 15, 2009 (KR) 10-2009-0003473
Aug. 18, 2009 (KR) 10-2009-0076437

(51) Int. Cl. A61B 5/145; G01N 27/416

(52) U.S. Cl. 600/345; 204/403.01

(57) ABSTRACT

Disclosed is a biosensor to which an electroactive polymer layer is attached, more specifically, a biosensor including an electroactive polymer layer attached to the surface of a bioreceptor and electrodes connected to the electroactive polymer layer, which allows reversible deformation of the electroactive polymer layer when an electrical stimulation is applied to the electrode and can thereby analyze the concentration of an analyte when the surface of the bioreceptor is exposed to the analyte. When used as an implantable biosensor, the disclosed biosensor is advantageous in that the time period for which and the frequency with which the bioreceptor is exposed to the analyte can be adjusted, and thus the lifespan of the biosensor is considerably extended.

### TECHNICAL FIELD

The present disclosure relates to a biosensor to which an electroactive polymer layer is attached, and more specifically, to a biosensor including an electroactive polymer layer attached to the surface of a bioreceptor and electrodes connected to the electroactive polymer layer, which allows reversible deformation of the electroactive polymer layer when an electrical stimulation is applied to the electrode and can thereby analyze the concentration of an analyte when the surface of the bioreceptor is exposed to the analyte.

### BACKGROUND ART

Biosensors for detecting analytes such as glucose for monitoring the status of a diabetic patient have been consistently studied for several decades. At present, disposable sensors are available. With the development of the sensor technology; researches are actively carried out to develop implantable biosensors allowing accurate and consistent measurement of the level of analytes in the body (see Jung et. al., Macromolecules 33, 3332-3336, 2000, Han et al., Biomacromolecules 3, 1271-1275, 2002. Wickramasinghe et al., Journal of Fluorescence 14, 513-520, 2004 and Koschwanez et al., Biomaterials 28, 3687-3703, 2007.).

The implantable biosensors developed thus far include, in addition to the biosensor in the most basic form for detecting electrochemical enzymatic reactions by the analyte, a biosensor measuring the change in output caused by the change in pH or pressure due to enzymes in a hydrogel depending on the change in analyte concentration (see PCT/US2000/23194 and PCT/US2001/12934), a transdermal biosensor based on reverse iontophoresis (Korean Patent No. 10-0541267), and so forth.

However, the existing biosensors share the technical limitation of short lifespan in the body regardless of the underlying principle. That is to say, since all the existing implantable biosensors are always exposed to the blood or body fluid, proteins or other disturbing substances existing in the blood or body fluid adhere onto the surface of the sensor or form films thereon, Consequently, the performance of the biosensor is degraded rapidly with time to an extent that it cannot perform as a biosensor.

### DISCLOSURE

### Technical Problem

The inventors of the present disclosure have found out that the problems of the existing implantable biosensor can be solved by attaching an electroactive polymer whose volume changes reversibly in response to an electrical stimulation on the surface of a biosensor and then selectively applying an electrical stimulation thereto.

The present disclosure is directed to providing a biosensor having an electroactive polymer attached on the surface of a bioreceptor, such that it allows the analysis of the analyte concentration by applying an electrical stimulation.

The present disclosure is also directed to providing an apparatus for analyzing the concentration of an analyte, using an implantable biosensor allowing selective concentration analysis.

The present disclosure is also directed to providing an implantable biosensor allowing selective concentration analysis.

The present disclosure is also directed to providing a method for selectively controlling the operation of an implantable biosensor by applying an electrical stimulation.

### Technical Solution

In one general aspect, the present disclosure provides a biosensor including: a bioreceptor capable of detecting an analyte to be analyzed; a signal transducer converting the concentration information of the analyte detected by the bioreceptor into an analyzable signal; an electroactive polymer layer attached on the surface of the bioreceptor; and an electrode connected to the electroactive polymer layer.

In an embodiment of the present disclosure, the electroactive polymer may contract and relax. More specifically, it may be an artificial muscle that contracts and relaxes.

In an embodiment of the present disclosure, the electroactive polymer may be an electroactive hydrogel.

In an embodiment of the present disclosure, the biosensor may be implantable.

In an embodiment of the present disclosure, the analyte may be glucose.

In another general aspect, the present disclosure provides an apparatus for analyzing the concentration of an analyte using an implantable biosensor, including: a biosensor implanted in the body of a patient, the biosensor including a bioreceptor capable of detecting an analyte to be analyzed; a signal transducer converting the concentration information of the analyte detected by the bioreceptor into an analyzable signal; an electroactive polymer layer attached on the surface of the bioreceptor; and an electrode connected to the electroactive polymer layer; a means for applying an electrical stimulation to the electrode connected to the electroactive polymer; a means for transmitting the concentration analysis information generated by the biosensor; and a computer means for receiving the concentration analysis information from the means for transmitting the information and outputting it.

In an embodiment of the present disclosure, the biosensor may be provided in plural numbers corresponding to the number of channels holding the analyte to be analyzed.

In another general aspect, the present disclosure provides a method for using a biosensor implanted in the body of a patient, including: providing a biosensor including a bioreceptor capable of detecting an analyte to be analyzed; a signal transducer converting the concentration information of the analyte detected by the bioreceptor into an analyzable signal; an electroactive polymer layer attached on the surface of the bioreceptor; and an electrode connected to the electroactive polymer layer; providing a computer means connected to the biosensor and outputting a concentration value of the analyte as a data signal; implanting the biosensor in the body of a patient and applying an electrical stimulation to the electrode connected to the electroactive polymer layer; and receiving the concentration information of the analyte detected by the bioreceptor via the computer means when the bioreceptor is exposed to the analyte as the electroactive polymer layer is reversibly deformed by the applied electrical stimulation, and decoding the received concentration information as a concentration value.

In another general aspect, the present disclosure provides a method for selectively controlling a biosensor implanted in the body of a patient, including: providing a biosensor including a bioreceptor capable of detecting an analyte to be analyzed; a signal transducer converting the concentration information of the analyte detected by the bioreceptor into an analyzable signal; an electroactive polymer layer attached on the surface of the bioreceptor; and an electrode connected to the electroactive polymer layer; implanting the biosensor in the body of a patient; selectively applying an electrical stimulation to the electrode connected to the electroactive polymer layer; and receiving the concentration information of the analyte detected by the bioreceptor when the bioreceptor is exposed to the analyte as the electroactive polymer layer is reversibly deformed by the applied electrical stimulation, and decoding the received concentration information as a concentration value.

In an embodiment of the present disclosure, the electroactive polymer layer may include a material that contracts and relaxes in response to the electrical stimulation.

### Advantageous Effects

Since the biosensor according to the present disclosure allows selective control of the time period for which and the frequency with which the bioreceptor is exposed to the analyte using the electroactive polymer, the durability and lifespan of the biosensor can be improved remarkably. Especially, since the contact between the surface of the biosensor and the analyte can be controlled through reversible deformation of the electroactive polymer in response to an electrical stimulation, the problem of decreased sensor lifespan caused by the adsorption of proteins on the surface of the implantable biosensor can be solved.

### DESCRIPTION OF DRAWINGS

FIG. 1 shows a cross-section of a biosensor according to an embodiment of the present disclosure and an operation mechanism thereof.

FIG. 2 shows a biosensor according to an embodiment of the present disclosure provided in a multichannel environment and an operation mechanism thereof.

FIG. 3 schematically shows a biosensor with an electroactive polymer attached thereto according to an embodiment of the present disclosure.

### DETAILED DESCRIPTION OF MAIN ELEMENTS

A: electroactive polymer
S: sensor
C: channel (body fluid/blood)
C_1: channel 1
C_2: channel 2
PS: power supply
CC: controller

### BEST MODE

Hereinafter, the embodiments of the present disclosure will be described in detail with reference to accompanying drawings.

As used herein, “analyte” refers to a chemical constituent to be analyzed. Although biomaterials such as glucose, DNA, enzyme, protein, cell, hormone, etc. are described as examples of the analyte, general chemical substances are not excluded from the analyte.

In the present disclosure, an electroactive polymer (EAP) is attached to a bioreceptor. The electroactive polymer is a material that can undergo reversible deformation in response to an external stimulation such as pH, solvent composition, ion concentration, electric field, or the like. Such a system that converts a chemical free energy into a mechanical work in response to a stimulation from its surroundings is called the ‘chemomechanical system’. The electroactive polymer is a polymer material belonging to the chemomechanical system that can contract and relax or move leftward and rightward using the chemical free energy in the polymer in response to an electrical stimulation. It is a kind of polymer hydrogel.

The electroactive polymers can be classified into ones in which actuation is caused by an electric field and those in which actuation is caused by ions. The electric field-based electroactive polymers can be classified into piezoelectric, electrostrictive and ferroelectric materials. The ionization-based electroactive polymers are deformed due to displacement of ions when an electric field is applied. Polymer gel and on film are examples. Besides, various forms of electroactive polymers including carbon nanotube, paper, cloth and fluid are studied.

Because of the advantages of being reversibly deformable (contraction/relaxation or leftward/rightward movement) in response to external stimulation, having high elasticity, being lightweight and being miniaturizable, the electroactive polymer can be developed into artificial muscles, small and noiseless actuators or biosensors capable of detecting various biological signals from the living body. Thus, it is expected to bring new technical innovation in many feature industries, including robotics, biology, aviation, space, military, and microelectromechanical systems (MEMS).

The electroactive polymer that can be used in the present disclosure may be any one that undergoes reversible volume change in response to electrical stimulation, especially one that can convert the change in chemical free energy in the polymer caused by electrical stimulation into mechanical work such as contraction or relaxation. In general, a material that can be used for artificial muscles may be used.

The electroactive polymers that can be used as artificial muscle materials include, dielectric elastomer actuators (DEA), relaxor ferroelectric polymers, and liquid-crystal rubbers. Further, ionic EAPs requiring ionic flow may be used as artificial muscle materials. The use of an electrolyte is essential for the movement of ions. When the electrolyte is liquid, it is called wet EAP. No special name is given when a solid electrolyte is used. When conducting polymer, carbon nanotube (CNT) or ionic polymer metal composite (IMPC) is used, the name of the material is used to denote the corresponding artificial muscle material.

In the present disclosure, an interpenetrating polymer network (IPN) hydrogel may be used as the electroactive polymer. The IPN refers to two or more networks which are at least partially interlaced on a polymer scale but not covalently bonded to each other. The network cannot be separated unless chemical bonds are broken. The IPNs are classified according to the polymerization method and type. Some IPNs form damping materials or reinforced elastomers of wide temperature range that can replace thermosetting resins. And, some IPNs exhibit continuous physical and mechanical properties that can hardly be attained with other polymers. The hydrogel refers to a network of hydrophilic polymer chains with low crosslinking density. Since it is a hydrated, crosslinked polymer system that can contain 20-90% of water in equilibrium state, it is permeable to oxygen and biocompatible. Since the IPN system is quick and sensitive to electrical stimulation and exhibits good mechanical properties (Kim et al, J. Appl. Polym. Sci., 73, 1675-1683, 1999), it can be effectively used in actuators, sensors and artificial muscles.

The operation mechanism of the biosensor according to the present disclosure will be described in detail referring to the attached drawings.

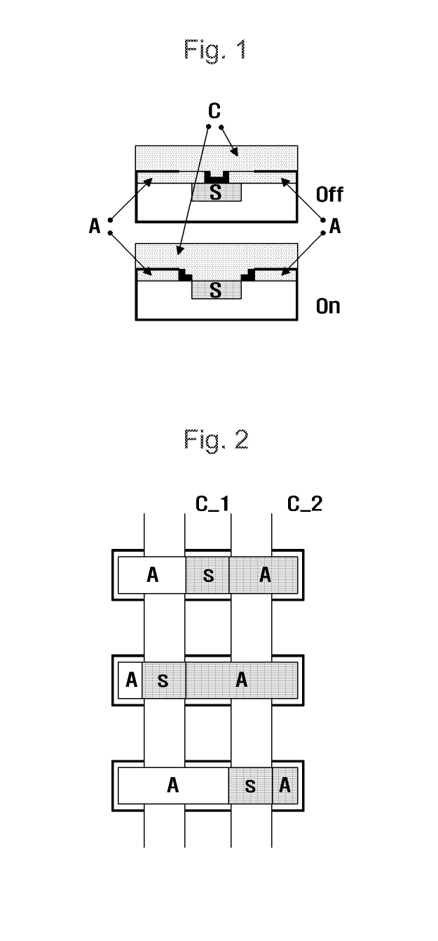

In the biosensor according to the present disclosure, a sensor S is selectively contacted with a channel C. The channel C may comprise either a single channel C or multiple channels (channel 1 C_1, channel 2 C_2, . . . ) (see FIG. 2).

An electroactive polymer A is attached to the sensor S of the implantable biosensor, and electrodes are connected to both ends of the electroactive polymer A in order to apply an electrical stimulation to the electroactive polymer A. The electrode may comprise an unharmful biocompatible material, and may be have a shape of needle, plate or disc. The electrode may comprise a single electrode or multiple electrodes arranged in array form attached or fixed to the electroactive polymer so as to apply the electrical stimulation.

FIG. 1 shows a cross-section of a biosensor according to an embodiment of the present disclosure and an operation mechanism thereof. When an electroactive polymer is attached to the biosensor, more specifically to a bioreceptor of the biosensor, and an electrical stimulation is applied to an electrode connected to the electroactive polymer, the electroactive polymer A exhibits reversible contraction/relaxation behavior. Thus, the surface of the biosensor S is exposed to a channel C and conies in contact with an analyte.

In FIG. 1, the OFF state at the top is a state wherein no electrical stimulation is applied, and the ON state at the bottom is a state wherein an electrical stimulation is applied and thus the biosensor S that has been covered by the electroactive polymer A is exposed as the electroactive polymer A contracts. That is to say, as the electroactive polymer A on the surface of the biosensor contracts in response to the electrical stimulation, the analyte (usually body fluid or blood) that has been covered by the electroactive polymer A is exposed to the biosensor S to allow the measurement of the analyte concentration.

FIG. 2 shows a biosensor according to an embodiment of the present disclosure provided in a multichannel environment and an operation mechanism thereof. As shown in FIG. 2, as the electroactive polymer A contracts, the first and seconds sensors may be exposed to a channel C_1, and the third sensor may be exposed to a channel C_2.

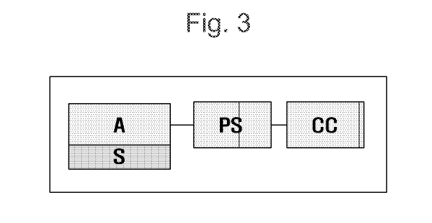

FIG. 3 schematically shows a biosensor with an electroactive polymer attached thereto according to an embodiment of the present disclosure. A power supply PS supplies power to the electroactive polymer A via an electrode, and a controller CC generates a power control signal and transmits it to the power supply PS for analysis of the analyte. When the power is turned off, the electroactive polymer A is set to such a position that the biosensor S is not exposed to the channel C for protection of the sensor.

For instance, when it is desired to analyze the analyte using the implantable biosensor 3, the controller CC generates a signal for controlling the electroactive polymer A and transmits it to the power supply PS. Then, the power supply PS supplies the power for controlling the electroactive polymer A. When the power is supplied in response to the control signal, the electroactive polymer A is reversibly deformed (i.e., contracts) and, as a result, the biosensor S is allowed to selectively contact with the analyte in the channel C.

The control signal from the implantable biosensor can be automatically transmitted to the controller CC of the biosensor. In response to the control signal, the controller CC transmits an operation signal to the power supply PS, thus allowing the control of power supply to the power supply PS and measurement of analyte concentration by the biosensor. Through such a control action, the biosensor may be allowed to contact with the blood or body fluid only for a minimum time period when the measurement is desired.

The biosensor according to an embodiment of the present disclosure may be provided as an apparatus for analyzing the concentration of an analyte together with a means for applying an electrical stimulation to the electrode connected to the electroactive polymer of the biosensor, a means for transmitting the concentration analysis information generated by the biosensor, and a computer means for receiving the concentration analysis information from the means for transmitting the information and outputting it.

A method for using an implantable biosensor with the electroactive polymer layer attached implanted in the body of a patient is as follows.

First, a biosensor to which an electroactive polymer layer exhibiting reversible deformation (particularly contraction) in response to an electrical stimulation is provided. Then, a computer means connected to the biosensor and outputting a concentration value of the analyte as a data signal is provided. The biosensor is implanted in the body of a patient and an electrical stimulation is applied to the electrode connected to the electroactive polymer layer. When the electroactive polymer layer is reversibly deformed by the applied electrical stimulation, the bioreceptor is exposed to the analyte and the concentration information of the analyte detected by the bioreceptor is received via the computer means. The received concentration information may be decoded as a concentration value.

The biosensor according to an embodiment of the present disclosure may be selectively controlled by applying an electrical stimulation. Details are as follows.

First, a biosensor to which an electroactive polymer layer exhibiting reversible deformation (particularly contraction) in response to an electrical stimulation is provided, and the biosensor is implanted in the body of a patient. Then, an electrical stimulation is selectively applied to the electrode connected to the electroactive polymer layer. When the electroactive polymer layer is reversibly deformed by the selectively applied electrical stimulation, the bioreceptor is exposed to the analyte and the concentration information of the analyte detected by the bioreceptor is received. The received concentration information may be decoded as a concentration value.

Through this mechanism, analysis of the analyte concentration by the biosensor can be achieved selectively by applying the electrical stimulation. Consequently, even when the biosensor is implanted in the body and used for a long period of time, the problem of deteriorated function and decreased sensor lifespan caused by the adsorption of proteins on the surface of the biosensor can be solved since the time period for which and the frequency with which the bioreceptor is exposed to the analyte can be reduced.

### MODE FOR INVENTION

The examples and experiments will now be described. The following examples and experiments are for illustrative purposes only and not intended to limit the scope of the present disclosure.

Although glucose is described as an example of analyte, those skilled in the art will understand that the scope of the present disclosure is not limited thereto.

### Example 1

### Control of Biosensor to which Electroactive Polymer is Attached Under Single-Channel Environment

First, control of an implantable biosensor to which an electroactive polymer is attached was tested under a single-channel environment.

For analysis of glucose in a single channel C using an implantable biosensor S, a signal for controlling an electroactive polymer A was generated at a controller CC and transmitted to a power supply PS. Thus, the power supply PS could supply the power for controlling the electroactive polymer A (see FIG. 1 and FIG. 3). As a result of the supply of power in response to the control signal, the electroactive polymer A contracted and the biosensor S was brought in contact with the glucose in the channel (see FIG. 1).

### Example 2

### Control of Biosensor to which Electroactive Polymer is Attached Under Multichannel Environment

Next, control of an implantable biosensor to which an electroactive polymer is attached was tested under a multichannel environment.

For analysis of glucose in multiple channels C_1, C_2 using an implantable biosensor S, a signal for controlling an electroactive polymer A was generated at a controller CC and transmitted to a power supply PS. Thus, the power supply PS could supply the power for controlling the electroactive polymer A (see FIG. 2 and FIG. 3). By generating different control signals for the channel 1 C_1 and the channel 2 C_2, the electroactive polymer A could be allowed to contract selectively. Consequently, the sensor S could be selectively brought in contact with the glucose in the 1 C_1 or with the glucose in the channel 2 C_2.

In accordance with the present disclosure, by selectively exposing the surface of a biosensor to the environment in the body using an electroactive polymer that contracts and relaxes in response to an electrical stimulation, the damage to the biosensor surface can be minimized and the lifespan of the biosensor can be maximized. For instance, when an analyte needs to be monitored every hour, by exposing the biosensor for only 1 minute at every hour, the surface damage or functional loss of the sensor can be reduced to 1/60 as compared to when the sensor surface is continuously exposed to the blood or body fluid. Therefore, the lifespan of the sensor may be increased by 60 times. Since the sensor lifespan can be improved remarkably regardless of the operation principle of the biosensor, the present disclosure will facilitate the use of the many implantable biosensors presented thus far.

Those skilled in the art will appreciate that the conceptions and specific embodiments disclosed in the foregoing description may be readily utilized as a basis for modifying or designing other embodiments for carrying out the same purposes of the present disclosure. Those skilled in the art will also appreciate that such equivalent embodiments do not depart from the spirit and scope of the disclosure as set forth in the appended claims.

1. A biosensor for analyzing the concentration of an analyte, comprising:
a bioreceptor capable of detecting an analyte to be analyzed;
a signal transducer converting the concentration information of the analyte detected by the bioreceptor into an analyzable signal;
an electroactive polymer layer attached on the surface of the bioreceptor; and
an electrode connected to the electroactive polymer layer.

2. The biosensor according to claim 1, wherein the electroactive polymer contracts and relaxes.

3. The biosensor according to claim 2, wherein the electroactive polymer is an artificial muscle that contracts and relaxes.

4. The biosensor according to claim 1, wherein the electroactive polymer is an electroactive hydrogel.

5. The biosensor according to claim 1, wherein the biosensor is implantable.

6. The biosensor according to claim 1, wherein the analyte is glucose.

7. An apparatus for analyzing the concentration of an analyte using an implantable biosensor, comprising:
a biosensor implanted in the body of a patient, the biosensor comprising a bioreceptor capable of detecting an analyte to be analyzed; a signal transducer converting the concentration information of the analyte detected by the bioreceptor into an analyzable signal; an electroactive polymer layer attached on the surface of the bioreceptor; and an electrode connected to the electroactive polymer layer;
a means for applying an electrical stimulation to the electrode connected to the electroactive polymer;
a means for transmitting the concentration analysis information generated by the biosensor; and
a computer means for receiving the concentration analysis information from the means for transmitting the information and outputting it.

8. The apparatus for analyzing the concentration of an analyte according to claim 7, wherein a plurality of the biosensors corresponding to the number of channels holding the analyte to be analyzed are provided.

9. A method for using a biosensor implanted in the body of a patient, comprising:
providing a biosensor comprising a bioreceptor capable of detecting an analyte to be analyzed; a signal transducer converting the concentration information of the analyte detected by the bioreceptor into an analyzable signal; are electroactive polymer layer attached on the surface of the bioreceptor; and an electrode connected to the electroactive polymer layer;
providing a computer means connected to the biosensor and outputting a concentration value of the analyte as a data signal;
implanting the biosensor in the body of a patient and applying an electrical stimulation to the electrode connected to the electroactive polymer layer; and
receiving the concentration information of the analyte detected by the bioreceptor via the computer means when the bioreceptor is exposed to the analyte as the electroactive polymer layer is reversibly deformed by the applied electrical stimulation, and decoding the received concentration information as a concentration value.

10. A method for selectively controlling a biosensor implanted in the body of a patient, comprising:
providing a biosensor comprising a bioreceptor capable of detecting an analyte to be analyzed; a signal transducer converting the concentration information of the analyte detected by the bioreceptor into an analyzable signal; an electroactive polymer layer attached on the surface of the bioreceptor; and an electrode connected to the electroactive polymer layer;
implanting the biosensor in the body of a patient;
selectively applying an electrical stimulation to the electrode connected to the electroactive polymer layer; and
receiving the concentration information of the analyte detected by the bioreceptor when the bioreceptor is exposed to the analyte as the electroactive polymer layer is reversibly deformed by the applied electrical stimulation, and decoding the received concentration information as a concentration value.

11. The method according to claim 9 or 10, wherein the electroactive polymer layer comprises a material that contracts and relaxes in response to the electrical stimulation.
